(19) United States
(12) Patent Application Publication
(10) Pub. No.: US 2024/0081840 A1
(43) Pub. Date: Mar. 14, 2024

(54) ORTHOPEDIC INSTRUMENT WITH INTERIOR CAVITY AND PASSAGES THERETO

(71) Applicant: ZIMMER, INC., Warsaw, IN (US)

(72) Inventors: Jason M. Endsley, Warsaw, IN (US); Tayler Kreider, Warsaw, IN (US); Steven Conrad, Albion, IN (US); William Paul Rodgers, III, Warsaw, IN (US)

(21) Appl. No.: 18/283,664

(22) PCT Filed: Apr. 20, 2022
(86) PCT No.: PCT/US2022/025580

§ 371 Date: Sep. 22, 2023

Related U.S. Application Data
(60) Provisional application No. 63/181,448, filed on Apr. 29, 2021.

(51) Int. Cl. A61B 17/16

(52) U.S. Cl. CPC A61B 17/164; A61B 17/1659; A61B 2017/00526

(57) ABSTRACT

Orthopedic instalments for removing bone including along an intramedullary canal and methods of their fabrication are disclosed. In one example, the orthopedic instrument can include a body having an exterior surface and can define an interior cavity. The body can have a plurality of channels communicating with the interior cavity from the exterior surface.

### CLAIM OF PRIORITY

This application claims the benefit of U.S. Provisional Patent Application Ser. No. 63/181,448, filed on Apr. 29, 2021, the benefit of priority of which is claimed hereby, and which is incorporated by reference herein in its entirety.

### FIELD OF THE DISCLOSURE

The present disclosure relates to orthopedic bone cutting instruments and methods relating to the fabrication thereof.

### BACKGROUND

Implantable orthopedic devices can replace or augment body components or portions of body components that cannot be regenerated or are no longer functioning properly. Examples of implantable orthopedic devices include hip replacement implants, knee replacement implants, spinal implants, dental implants, and other joint implants.

Preparation of the body to receive implantable orthopedic devices is a complex process that utilizes many specifically adapted instruments. Instruments such as broaches, rasps and reamers are utilized to cut and remove bone and/or soft tissue in preparation for receiving the implantable orthopedic device.

### Overview

Various embodiments discussed in the present document relate to orthopedic bone cutting instruments such as, but not limited to, rasps, broaches and/or reamers. The present inventors recognize that orthopedic bone cutting instruments can include features such as cutting teeth that can become clogged with bone debris as a result of use. For example, femoral rasps typically compact bone debris during impaction clogging the teeth and making the rasp less effective. This clogging increases the required impaction cycles and forces. Higher impaction forces can increase the risk for an intraoperative femoral fracture. To alleviate this risk, it is suggested that rasps be intermittently removed from the bone during the surgery and the teeth thereto cleaned of debris before impaction into bone resumes. This intra-operative cleaning of the rasp can increase surgical time.

The present inventors recognize orthopedic bone cutting instruments such as those disclosed herein can be configured to reduce the clogging of teeth with bone debris. To this end, the present inventors have designed an orthopedic bone cutting instrument that allows bone debris to flow away from the teeth through a wall or body of the instrument to a hollow center of the instrument. This configuration of the instrument allows the instrument, such as a rasp, to progress with less axial force, and hence, less radial force, reducing the risk of bone fracture. The configuration also reduces the number of strikes needed by the surgeon to seat the rasp at a desired level. Hence, the design reduces the strain on the surgeon, the impaction time or both. The design also reduces the need for intra-operative cleaning of the instrument as discussed previously.

The orthopedic instruments discussed and illustrated herein can be fabricated using additive manufacturing techniques. Such additive manufacturing techniques make it more feasible and more cost effective for the orthopedic instruments to have interior features such as the cavity and/or the channels.

### BRIEF DESCRIPTION OF THE FIGURES

FIG. 1 is a plan view of an orthopedic instrument according to an example of the present application.

FIG. 2 is a schematic view of the orthopedic instrument of FIG. 1 according to an example of the present application.

FIG. 2A is a cross-sectional view of the orthopedic instrument of FIG. 2.

FIG. 2B is an enlarged cross-sectional view of a portion of the orthopedic instrument of FIG. 2.

FIG. 3 is an enlarged view of a tooth and opening pattern for an orthopedic instrument according to another example of the present application.

FIG. 3A is a cross-sectional view of the orthopedic instrument of FIG. 3.

FIG. 3B is an enlarged view of several of the teeth of the orthopedic instrument of FIG. 3.

FIGS. 4A and 4B are plan views of an orthopedic instrument according to yet another example of the present application.

FIG. 4C is an enlarged view of a portion of the orthopedic instrument of FIGS. 4A and 4B.

FIG. 5 is a perspective view of yet another an orthopedic instrument according to yet another example of the present application.

FIG. 5A is an enlarged view of the orthopedic instrument of FIG. 5.

### DETAILED DESCRIPTION

Orthopedic bone cutting instruments such as rasps, broaches and/or reamers are used to prepare bone including the intramedullary canal. For example, a femoral rasp can be used to prepare the femur including the intramedullary canal thereof. The prepared canal determines the fit between the prosthetic femoral hip stem and the femur, which in turn determines the fit between the prosthetic femoral head and the acetabulum. Similarly, a broach can be used to prepare the intramedullary canal of a humerus during a shoulder replacement procedure. Reamers are utilized to prepare the intramedullary canal of the tibia during a knee replacement procedure. Although exemplary designs are described herein in reference to a rasp, such a femoral rasp configured to prepare a cavity in a femur, the present invention is generally applicable to any orthopedic bone cutting device configured to prepare a cavity in bone, such as bone of a tibia, femur, humerus, etc.

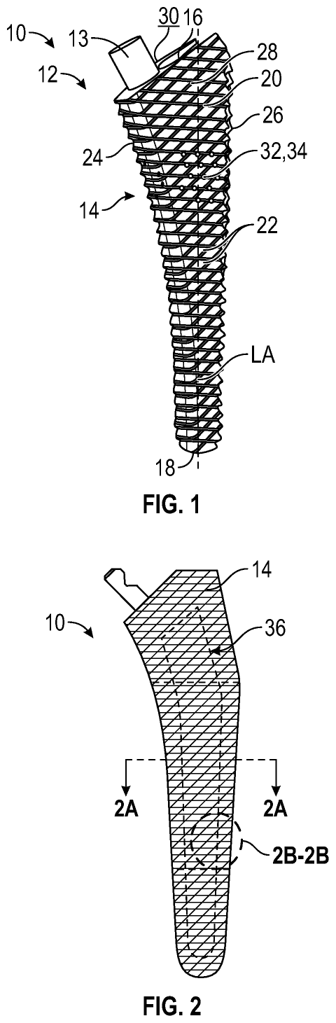

FIG. 1 shows an orthopedic instrument 10 comprising a rasp 12 such as a femoral rasp. The rasp 12 can include a handle 13 and an elongate body 14. The elongate body 14 has a shape that generally corresponds to the geometry of prosthetic femoral hip stem to be implanted into the femur.

The handle 13 can connect with the elongate body 14 at a proximal end thereof. The handle 13 can be configured to facilitate grasping and manipulation of the elongate body 14 by the surgeon. The handle 13 and the elongate body 14 can be formed as a single, unitary structure, or the handle 13 can be a separate piece that is attached to the elongate body 14.

The elongate body 14 has a longitudinal axis LA and extends between proximal end 16 and distal end 18. The elongate body 14 can narrow from proximal end 16 to distal end 18. The elongate body 12 can additionally form an exterior surface 20 of the rasp 12. The elongate body 12 that forms the exterior surface 20 can have various features such as a plurality of teeth 22 as further discussed herein.

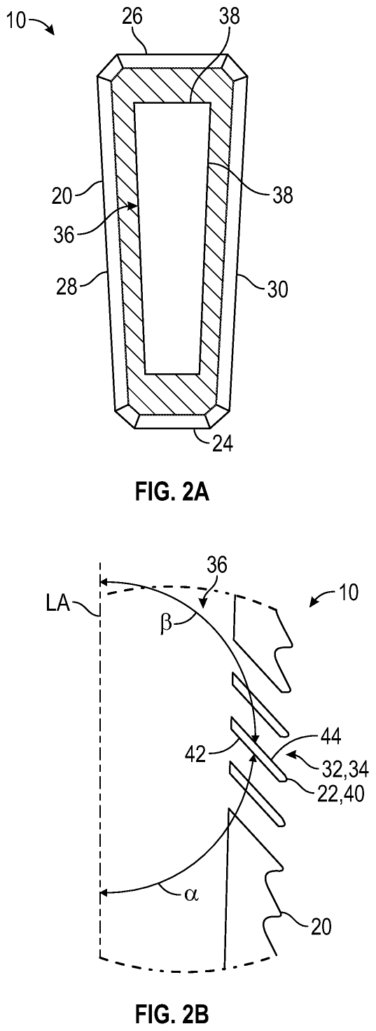

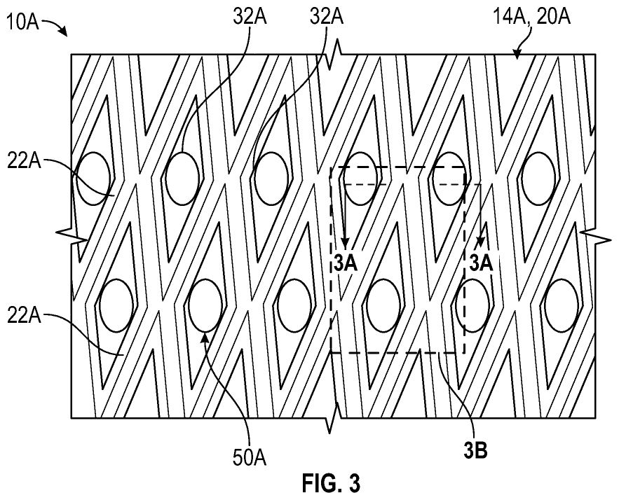

The elongate body 14 that forms the exterior surface 20 can be shaped at a macro-level to provide for a medial face 24, a lateral face 26, an anterior face 28, and a posterior face 30 according to one example. As used herein, anterior, posterior, lateral, and medial are determined by the intended use of the rasp to a person having ordinary skill in the art. These faces 24, 26, 28 and 30 can in combination form a generally trapezoidal cross-sectional shape for the body 14 such as shown in FIG. 2A. However, other cross-sectional shapes for the body 14 such as round or rectangular shapes are also contemplated. The medial face 24 can generally extend parallel to lateral face 26. Similarly, the anterior face 28 can extend generally parallel with or can converge toward the posterior face 30. The width of elongate body 14, or the distance between medial face 24 and lateral face 26 can exceed the depth of elongate body 14, or the distance between anterior face 28 and posterior face 30.

The plurality of teeth 22 and other features can form the exterior surface 20 at a more micro-scale than the macro-scale faces 24, 26, 28 and 30. The plurality of teeth 22 can extend from the elongate body 14, and more specifically from the medial face 24, the lateral face 26, the anterior face 28, and/or the posterior face 30 of the elongate body 14. The plurality of teeth 22 can extend a distance outward from elongate body 14 until reaching a tip (also sometimes referred to as an edge). The tip can be at a location furthest from the corresponding face of elongate body 14. The plurality of teeth 22 can also include roots. One or more roots can space an individual one of the plurality of teeth 22 from adjacent teeth. In some cases, the plurality of teeth 22 can extend a distance into the elongate body 14 or face. This inward distance can be the root. Various designs, shapes and sizes of teeth are contemplated as known in the art. The plurality of teeth 22 can have an outward extent of as large as approximately 0.5 millimeter to 1.5 millimeters, inclusive, for example. Adjacent of the plurality of teeth 22 can be spaced apart by a distance as measured between the tips. This spacing distance can be as small as approximately 1.0 millimeter or as large as approximately 3.0 millimeters, for example. The depth of the plurality of teeth 22 and the spacing distance of the plurality of teeth 22 impact the amount of bone cut away by the plurality of teeth 22. Various different tooth shapes, spacing distances, tooth depths are contemplated herein.

FIG. 1 additionally illustrates some openings 32 of some a plurality of channels 34 at the exterior surface 20. These openings 32 can be adjacent the plurality of teeth 22 such as at the roots thereof as further discussed and illustrated herein. For simplicity, only some of the plurality of openings 32 and some of the plurality of channels 34 are shown in FIG. 1. These are shown on parts of the medial face 24, the lateral face 26, the anterior face 28. It is understood that further areas, parts of faces or other features of the elongate body 14 not specifically shown as having openings 32 and the plurality of channels 34 can have them according to other examples. The present application contemplates various design choices such as the number, size, shape, location, etc. of the openings 32 and the plurality of channels 34 can be modified as desired according to design, operation and other criteria. Thus, the number, size, shape and location of the openings 32 and the plurality of channels 34 of FIG. 1 is illustrated purely for exemplary purposes. The openings 32 and the plurality of channels 34 can be spread across substantially an entirety of the faces 24, 26, 28 and 30 according to some examples. According to other examples, the openings 32 and the plurality of channels 34 can be located only at certain areas or regions of the exterior surface 20.

FIG. 2 shows a schematic view of the orthopedic instrument 10 and further illustrates an interior cavity 36 within the elongate body 14 in phantom. FIG. 2A shows a cross-section of the orthopedic instrument 10 that extends through the cavity 36. FIGS. 2 and 2A do not specifically illustrate the openings 32 and the plurality of channels 34 (FIG. 1). However, it is understood that the plurality of channels 34 extend inward from the openings 32 to communicate with the cavity 36 as further illustrated in the enlarged cross-sectional view of FIG. 2B, for example.

As shown in FIG. 2A, the cavity 36 can be defined by interior surface(s) 38 of the elongate body 14. As shown in FIG. 2A, the interior surface 38 of the elongate body 14 that defines the interior cavity 36 can extend generally parallel with but is spaced from the exterior surface 20 (i.e. the faces 24, 26, 28 and 30). The interior cavity 36 can be entirely encapsulated by the elongate body 14 save for the plurality of channels 34 according to some examples. Thus, the elongate body 14 can comprise a hollow shell. The interior cavity 36 can have a volume of between 20% and 90% of a total volume of the elongate body 14, for example. However, the volume of the cavity 36 can be relatively larger or smaller according to further examples. The cavity 36 is shown in FIG. 2 as a single continuous volume. However, according to further examples the elongate body 14 can define two or more separate interior cavities. Additionally, details such as relief passages or openings for the removal of bone chips and to facilitate cleaning of the cavity 36 are contemplated but are not specifically illustrated in FIGS. 2 and 2A.

According to one example, the elongate body 14 can be fabricated using an additive manufacturing technique, such as 3D printing, selective laser sintering (SLS), selective laser melting (SLM), Rapid Prototyping (RP), Direct Digital Manufacturing (DDM), and/or any other additive manufacturing technique. According to further examples, the elongate body 14 can be formed through die casting or injection molding.

According to one example, it is contemplated that the elongate body 14 can be fabricated with features such as the cavity 36 and the plurality of channels 34 using binder jetting 3D printing. With binder jetting printing, a binder is selectively deposited onto the powder bed, bonding these areas is performed to bind these areas together to form a solid part one layer at a time. The materials contemplated for the elongate body 14 can be metals or polymeric metal powders, for example. According to one example, with binder jetting printing, a recoating blade spreads a thin layer of powder over a build platform to build a bed. A carriage with inkjet nozzles can pass over the bed, selectively depositing droplets of the binder that bonds the powder particles together. When the layer is complete, the build platform moves downwards and the blade re-coats the surface. The process then repeats until the elongate body 14 is completed. Curing, sintering and other additionally processing can then take place. In certain embodiments, the elongate body 14 can be formed using cross-sections generated from a 3-D digital description of the instrument, e.g., from a CAD file or scan data, on the surface of the powder bed. Net shape and near net shape constructs can be infiltrated and coated in some instances.

Thus, according to one example the orthopedic instrument 10 can be produced by a method that includes additively fabricating the elongate body 14 with a three-dimensional shape. The additively fabricating the elongate body 14 forms one or more features (e.g. openings 32, teeth 22, faces, etc.) at the exterior surface 20 of the body 14 including the plurality of teeth 22. The method can include additively fabricating the body 14 to form the body 14 as a shell about a cavity 36. The additively fabricating the 14 body can form the plurality of channels 34 extending through the body 14 to communicate with the cavity 36 from the exterior surface 20.

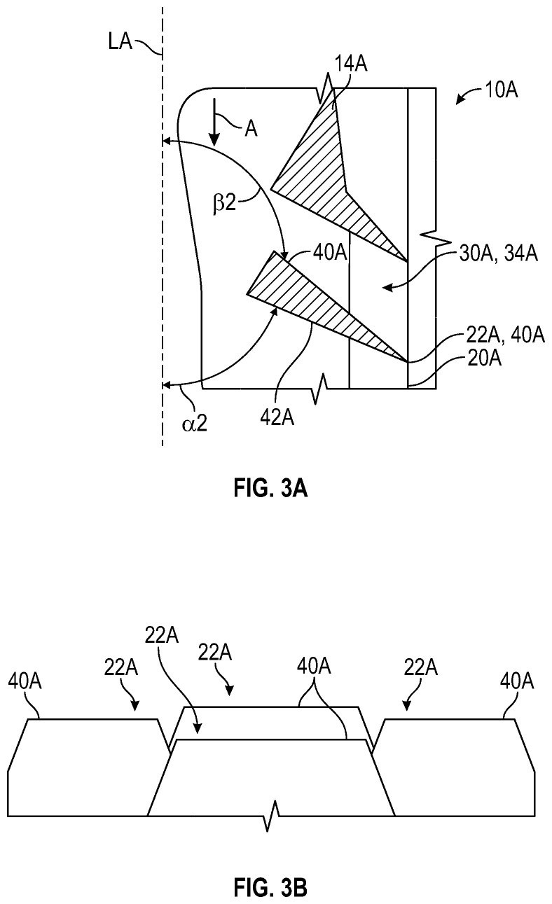

FIG. 2B shows an enlarged cross-section of a portion of the orthopedic instrument 10. FIG. 2B shows several of the plurality of passages 34 extending from the cavity 36 to the exterior surface 20 at openings 32. As shown in FIG. 2B, the openings 32 of the plurality of channels 34 to the exterior surface 20 can be spaced from tips 40 of the plurality of teeth 22. Put another way, the openings 32 of the plurality of channels 34 to the exterior surface 20 can be at the roots or another part of the plurality of teeth 22 as shown in FIGS. 1 and 3.

The plurality of channels 34 can be configured (shaped, sized, positioned, angled, etc.) to receive and communicate bone chips to the interior cavity 36. As shown in FIG. 2B, the plurality of teeth 22 can be canted (angled) relative to the longitudinal axis LA of the instrument 10 so that each of the plurality of teeth 22 have a first face 42 that forms an acute angle α to the longitudinal axis LA and a second opposing face 44 that forms an obtuse angle β to the longitudinal axis LA. This angulation can also be relative to a cutting or impaction direction of the instrument 10. Additionally, as show in FIG. 2B, the plurality of channels 34 can be canted (angled) relative to the longitudinal axis LA such that the orthopedic instrument 10 is configured with the plurality of channels 34 that are angled relative to the exterior surface 20 as defined tangent to the tips 40 of the plurality of teeth 22. This angle of the plurality of channels 34 can better facilitate receiving bone chips that result from insertion of the orthopedic instrument 10 into the bone.

FIG. 3 shows a portion of a second orthopedic instrument 10A having a different shape and arrangement of a plurality of teeth 22A at an exterior surface 20A than those illustrated previously. As shown in FIG. 3, the openings 32A can be located at roots 50 between the plurality of teeth 22A. FIG. 3A shows a cross-section through the body 14A bisecting two of the plurality of teeth 22A and bisecting a couple of the plurality of channels 34A that communicate with the openings 32A.

As shown in FIG. 3A, the plurality of teeth 22A can be canted (angled) relative to the longitudinal axis LA and/or the cutting direction (indicated by arrow A) of the instrument 10. As a result of this arrangement each of the plurality of teeth 22A have a first face 42A that forms an acute angle α2 to the longitudinal axis LA and a second opposing face 44A that forms an obtuse angle β2 to the longitudinal axis LA. Additionally, as show in FIG. 3A, the plurality of channels 34A can be canted (angled) relative to the longitudinal axis LA and/or the cutting direction (indicated by arrow A) such that the orthopedic instrument 10A is configured with the plurality of channels 34A that are angled relative to the exterior surface 20A as defined tangent to tips 40A of the plurality of teeth 22A. This angle of the plurality of channels 34A can better facilitate receiving bone chips that result from insertion of the orthopedic instrument 10A into the bone (as indicated by arrow A).

FIG. 3B shows an enlarged view of several of the teeth 22A of the second orthopedic instrument 10A. FIG. 3B is a perspective view from a distal perspective looking toward a proximal end of the second orthopedic instrument 10A. Openings 32A (FIGS. 3 and 3B) are not visible in FIG. 3B due to the shape and orientation of the teeth 22A. FIG. 3B rather illustrates the tips 40A of the several teeth 22A. As shown in FIG. 3B, the teeth 22A do not have triangular pointed tips but rather can have a plateau, trapezoidal or mesa shape in profile. This shape can provide a substantially flat extent for the tip 40A. This substantially flat extent can extend along a medial-lateral circumference of the second orthopedic instrument 10A, for example. However, other orientations (e.g., proximal-distal, etc.) are contemplated according to other examples. The shape of the teeth 22A as shown in FIG. 3B can reduce hoop stress on the second orthopedic instrument 10A as compared with conventional pointed tipped teeth.

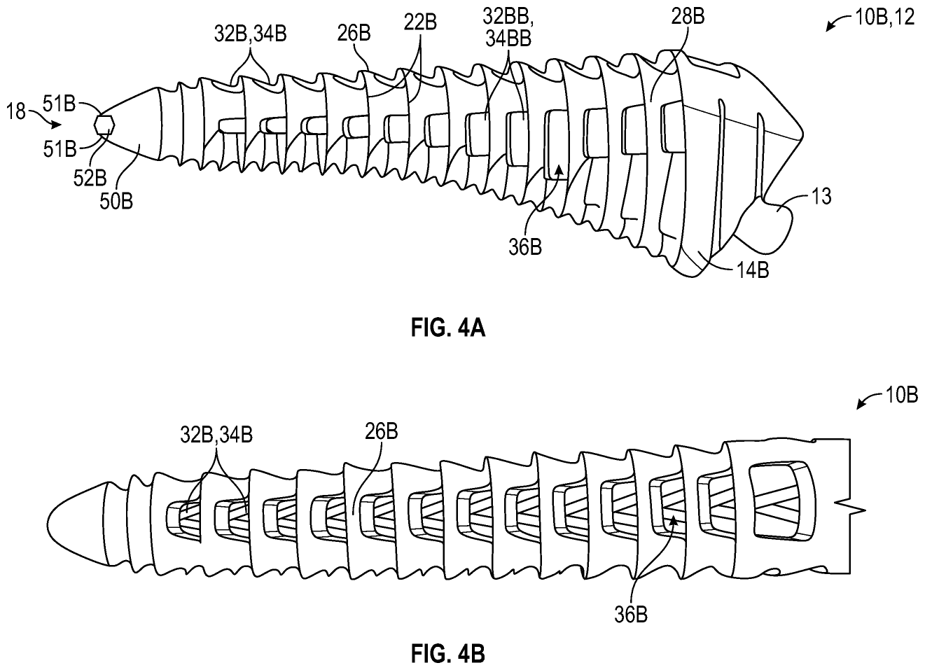

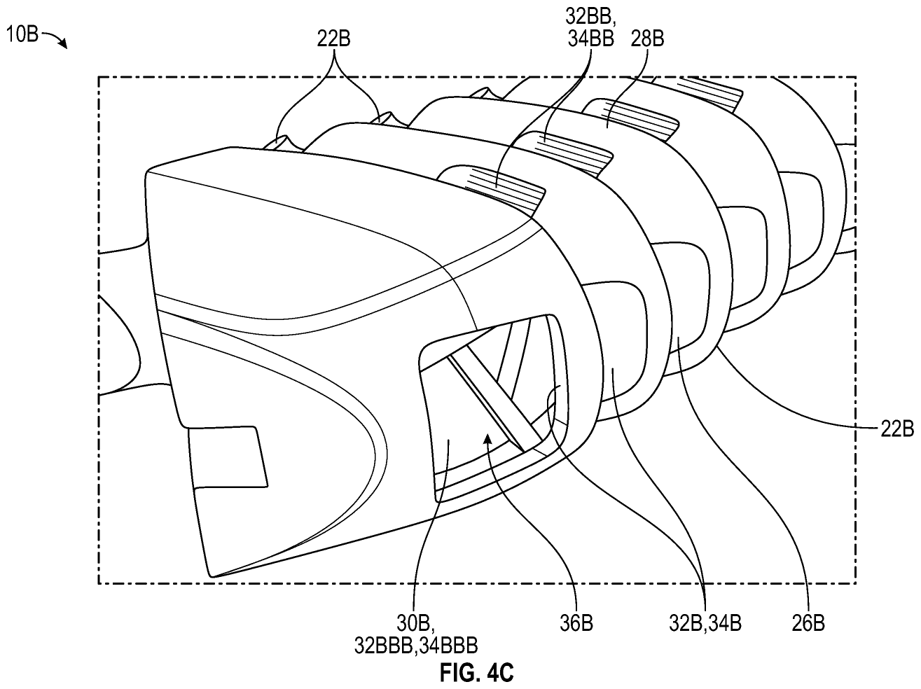

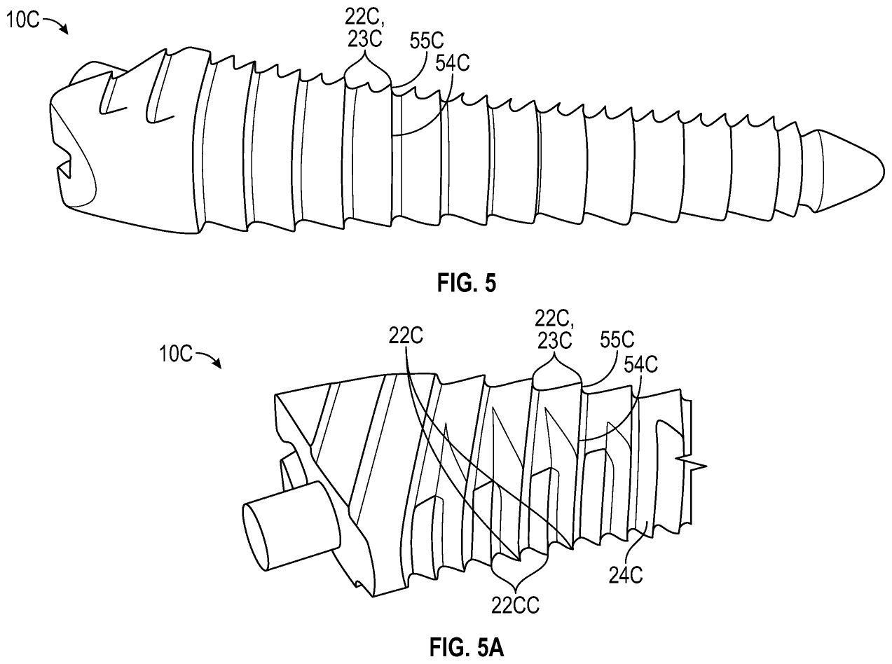

FIGS. 4A-4C show a third orthopedic instrument 10B. As shown in FIG. 4A, the orthopedic instrument can be shaped as the rasp 12 such as a femoral rasp. The rasp 12 can include the handle 13 and the elongate body 14B. The elongate body 14 has a shape that generally corresponds to the geometry of prosthetic femoral hip stem to be implanted into the femur.

Turning to FIGS. 4A-4C, the orthopedic instrument 10B can be constructed in a manner similar to that of the orthopedic instruments 10 and 10A previously described. However, the orthopedic instrument 10B can differ in some respects from those previously described. More particularly, the orthopedic instrument 10B can include at least the lateral face 26 having openings 32B and a plurality of channels 34B therein. The openings 32B via the plurality of channels 34B communicate with a cavity 36B defined within the elongate body 14B of the orthopedic instrument 10B. The openings 32B can be spaced by teeth 22B.

The anterior face 28B and/or the posterior face 30B can also include openings. In particular, FIG. 4A shows the anterior face 28B with openings 32BB and a plurality of channels 34BB. The openings 32BB via the plurality of channels 34BB communicate with the cavity 36B. FIG. 4C shows the posterior face 30B can include openings 32BBB and a plurality of channels 34BBB. The openings 32BBB via the plurality of channels 34BBB communicate with the cavity 36B.

The openings 32B, 32BB and/or 32BBB differ from the openings previously discussed in that the openings 32B, 32BB and/or 32BBB are much larger (e.g., having a major dimension of between 5 mm and 50 mm, inclusive). One or more of the openings 32B, 32BB and/or 32BBB can extend from the root of an associated tooth to within between 1.5 mm and 20 mm of a crest of an adjacent most tooth. The size of the openings 32B, 32BB and/or 32BBB relative to a total surface area of an exterior of the orthopedic instrument 10B can vary as desired. According to one example, the openings 32B can each be between 0.25% and 2.5% of the total surface area of the exterior of the orthopedic instrument 10B. The openings 32BB and/or 32BBB can be constructed in a similar manner to the openings 32B and can have a comparable size (e.g., can individually be between 0.25% and 2.5% of the surface area of the exterior of the orthopedic instrument 10B). Collectively the openings 32B, 32BB and/or 32BBB can be between 15% and 40% of the total surface area of the exterior of the orthopedic instrument 10B. Due to the size of the openings 32B, 32BB and/or 32BBB, the cavity 36 can be readily accessible to receive tissue via the openings 32B, 32BB and/or 32BBB and the plurality of channels 34B, 34BB and 34BBB.

Referring now to FIG. 4A, the orthopedic instrument 10B can also differ from the prior instruments in that the distal end 18 can be shaped as a punch 50B having a tip 51B with one or more openings 52B therein. The punch 50B with the tip 51B and tapered shape can be configured to cut through tissue and the tissue can be received by the one or more openings 52B in the punch 50B. The one or more openings 52B can communicate with the cavity 36B.

FIGS. 5 and 5A show a fourth orthopedic instrument 10C. The orthopedic instrument 10C can have similar construction to those previously discussed but may not include openings or a cavity therein. The orthopedic instrument 10C can include teeth 22C having an undercut geometry. This can provide the teeth 22C with a wave-like profile 23C as shown in FIGS. 5 and 5A. The undercut geometry can be aided or facilitated by one or more roots 54C along the surface(s) of the orthopedic instrument 10C. The one or more roots 54C can be grooves, troughs, radius or other feature positioned adjacent the face of one or more of the teeth 22C. Due to the undercut geometry, a tip 55C (or crest with the wave-like shape) of one or more of the teeth 22C can project over (e.g., extend distally over) an adjacent more distal roots 54C of an adjacent more distal tooth.

As best shown in FIG. 5A, smaller-height teeth 22CC can be added to the medial face 24C. These smaller-height teeth 22CC can not only have a relatively smaller height (e.g., between about 25% and about 75% of the height of the teeth 22C) but can also have a tighter radius shape for a better tolerance clearing of tissue to create a cavity in the tissue. The relatively larger height teeth 22C can be used in areas of the face/surface where it is desirable to clear tissue debris without as much concern for tolerance.

Reference is made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” “generally” or “substantially” or variations thereof as used herein can allow for a degree of variability in a value or range, for example, within 10%, within 5%, or within 1% of a stated value or of a stated limit of a range, and includes the exact stated value or range. The term “about” “generally” or “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Exemplary Embodiments

The following exemplary embodiments are provided, the numbering of which is not to be construed as designating levels of importance:

Example 1 is an orthopedic instrument that can include a body having an exterior surface and can define an interior cavity. The body can have a plurality of channels communicating with the interior cavity from the exterior surface.

Example 2 is the orthopedic instrument of Example 1, wherein the exterior surface can be defined at least partially by a plurality of teeth.

Example 3 is the orthopedic instrument of Example 2, wherein openings of the plurality of channels to the exterior surface can be at roots of the plurality of teeth.

Example 4 is the orthopedic instrument of Example 2, wherein openings of the plurality of channels to the exterior surface can be spaced from tips of the plurality of teeth.

Example 5 is the orthopedic instrument of any one of Examples 1-4, wherein the plurality of teeth can be canted relative to a longitudinal axis of the instrument so that each of the plurality of teeth have a first face that forms an acute angle to the longitudinal axis and a second opposing face that forms an obtuse angle to the longitudinal axis.

Example 6 is the orthopedic instrument of any one of Examples 1-5, wherein an interior surface of the body that defines the interior cavity can extend generally parallel with but is spaced from the exterior surface.

Example 7 is the orthopedic instrument of any one of Examples 1-6, wherein the plurality of channels can be configured to receive and communicate bone chips to the interior cavity.

Example 8 is the orthopedic instrument of any one of Examples 1-7, wherein the interior cavity can have a volume of between 20% and 90% of a total volume of the body.

Example 9 is the orthopedic instrument of any one of Examples 1-8, wherein the plurality of channels can be canted relative to a longitudinal axis such that the plurality of channels are angled relative to the exterior surface as defined tangent to tips of the plurality of teeth to receive bone chips that result from insertion of the orthopedic instrument into the bone.

Example 10 is a method of producing an orthopedic instrument that can include additively fabricating a body with a three-dimensional shape. The additively fabricating the body can form one or more features at an exterior surface of the body including a plurality of teeth. The method can further include additively fabricating the body to form the body about a cavity. Additively fabricating the body can form a plurality of channels extending through the body to communicate with the cavity from the exterior surface.

Example 11 is the method of Example 10, wherein fabricating one or more features at the exterior surface includes positioning openings of the plurality of channels to the exterior surface at roots of the plurality of teeth.

Example 12 is the method of Example 10, wherein fabricating one or more features at the exterior surface includes positioning openings of the plurality of channels to the exterior surface spaced from tips of the plurality of teeth.

Example 13 is the method of any one of Examples 10-12, wherein fabricating one or more features at the exterior surface includes canting the plurality of teeth relative a longitudinal axis of the instrument so that each of the plurality of teeth have a first face that forms an acute angle to the longitudinal axis and a second opposing face that forms an obtuse angle to the longitudinal axis.

Example 14 is the method of any one of Examples 10-13, wherein the additively fabricating the body provides the cavity has a volume of between 20% and 90% of a total volume of the body.

Example 15 is method of any one of Examples 10-14, wherein the additively fabricating the body includes canting the plurality of channels relative to a longitudinal axis such that the plurality of channels are angled relative to the exterior surface as defined tangent to tips of the plurality of teeth.

Example 16 is the method of any one of Examples 10-15, wherein additively fabricating the body includes binder jet printing the body.

Example 17 is any combination of the above Examples or parts/elements of the above Examples.

1. An orthopedic instrument for removing bone including along an intramedullary canal, the orthopedic instrument comprising:
a body having an exterior surface and defining an interior cavity, wherein the body has a plurality of channels communicating with the interior cavity from the exterior surface.

2. The orthopedic instrument of claim 1, wherein the exterior surface is defined at least partially by a plurality of teeth.

3. The orthopedic instrument of claim 2, wherein openings of the plurality of channels to the exterior surface are at roots of the plurality of teeth.

4. The orthopedic instrument of claim 2, wherein openings of the plurality of channels to the exterior surface are spaced from tips of the plurality of teeth.

5. The orthopedic instrument of claim 3, wherein the plurality of teeth are canted relative to a longitudinal axis of the instrument so that each of the plurality of teeth have a first face that forms an acute angle to the longitudinal axis and a second opposing face that forms an obtuse angle to the longitudinal axis.

6. The orthopedic instrument of claim 1, wherein an interior surface of the body that defines the interior cavity extends generally parallel with but is spaced from the exterior surface.

7. The orthopedic instrument of claim 1,
wherein the plurality of channels are configured to receive and communicate bone chips to the interior cavity.

8. The orthopedic instrument of claim 1, wherein the interior cavity has a volume of between 20% and 90% of a total volume of the body.

9. The orthopedic instrument of claim 1, wherein the plurality of channels are canted relative to a longitudinal axis such that the plurality of channels are angled relative to the exterior surface as defined tangent to tips of the plurality of teeth to receive bone chips that result from insertion of the orthopedic instrument into the bone.

10. A method of producing an orthopedic instrument comprising:
additively fabricating a body with a three-dimensional shape, wherein the additively fabricating the body forms one or more features at an exterior surface of the body including a plurality of teeth; and
additively fabricating the body to form the body about a cavity, wherein additively fabricating the body forms a plurality of channels extending through the body to communicate with the cavity from the exterior surface.

11. The method of claim 10, wherein fabricating one or more features at the exterior surface includes positioning openings of the plurality of channels to the exterior surface at roots of the plurality of teeth.

12. The method of claim 10, wherein fabricating one or more features at the exterior surface includes positioning openings of the plurality of channels to the exterior surface spaced from tips of the plurality of teeth.

13. The method of claim 10, wherein fabricating one or more features at the exterior surface includes canting the plurality of teeth relative a longitudinal axis of the instrument so that each of the plurality of teeth have a first face that forms an acute angle to the longitudinal axis and a second opposing face that forms an obtuse angle to the longitudinal axis.

14. The method of claim 10, wherein the additively fabricating the body provides the cavity has a volume of between 20% and 90% of a total volume of the body.

15. The method of claim 10, wherein the additively fabricating the body includes canting the plurality of channels relative to a longitudinal axis such that the plurality of channels are angled relative to the exterior surface as defined tangent to tips of the plurality of teeth.

16. The method of claim 10, wherein additively fabricating the body includes binder jet printing the body.
